(19) United States
(12) Patent Application Publication
(10) Pub. No.: US 2026/0318812 A1
(43) Pub. Date: Oct. 15, 2026

(54) BIOLOGICAL MEASUREMENT APPARATUS

(71) Applicant: Cytomos Limited, Edinburgh (GB)

(72) Inventor: Thomas CLAYTON, Edinburgh (GB)

(21) Appl. No.: 19/479,168

(22) PCT Filed: Apr. 26, 2024
(86) PCT No.: PCT/GB2024/051121

§ 371 Date: Oct. 27, 2025

(30) Foreign Application Priority Data
Apr. 28, 2023 (GB) 2306372.0

(51) Int. Cl. G01N 15/1031; G01N 33/487

(52) U.S. Cl. CPC G01N 15/1031; G01N 33/48707

(57) ABSTRACT

A biological measurement apparatus (10) comprises a stimulation apparatus (12) including at least one stimulation electrode (102a, 102b) and a stimulation circuit. The stimulation circuit is configured to provide an electric stimulus signal to the at least one stimulus electrode (102a, 102b), such that the at least one stimulus electrode (102a, 102b) generates a pre-determined, electrodynamic field that is applied to biological matter. The sensing apparatus includes at least one sensing electrode (202a, 202b) and a sensing circuit, the at least one sensing electrode (202a, 202b) configured to sense the response electrodynamic field and provide a corresponding electric response signal to the sensing circuit. The electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

The present invention relates to biological measurement apparatus.

The analysis of physical or chemical characteristics of biological particles finds use in the diagnosis of disease, in research, in clinical trials of drugs, and the like. It is known to employ measurement apparatus to carry out such analysis on an automatic basis with the apparatus being configured to provide for simultaneous multi-parametric measurement of particles. Typically, the biological particles of interest are suspended in a fluid before being introduced to the measurement apparatus where the suspended biological particles are subject to excitation and the response to the excitation is detected.

One example of such measurement apparatus utilises impedance or dielectric spectroscopy to measure characteristics of biological particles. Impedance or dielectric spectroscopy involves applying an electrodynamic field to a solution containing a biological particle and measuring the changes to the field due to the presence of the biological particle, eg caused by the complex electrical permittivity of a particle. Conventionally, impedance and dielectric spectroscopy apparatus use lock-in amplifiers to calculate the magnitude and phase shift of a signal caused by changes in the solution passing through a target region; such as the sudden presence of a cell. This means that for each such lock-in amplifier, the typical measurement apparatus can measure the impedance of a single frequency at a time.

Apparatus for measuring the impedance of many frequencies at a time, in order to analyse many different characteristics of a biological particle at a time, has been proposed, for example in WO 2015/001355. However, there are significant challenges in achieving high bandwidth in such measurement apparatus.

There have now been devised improved biological measurement apparatus which overcome or substantially mitigate the aforementioned and/or other disadvantages associate with the prior art.

According to a first aspect of the present invention, there is provided biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the at least one stimulus electrode generates a pre-determined, electrodynamic field that is applied to biological matter, and
a sensing apparatus including at least one sensing electrode and a sensing circuit, the at least one sensing electrode configured to sense the response electrodynamic field and provide a corresponding electric response signal to the sensing circuit,
wherein the electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

According to a further aspect of the present invention, there is provided a method of biological measurement, the method comprising:
providing an electric stimulus signal to at least one stimulus electrode, such that the at least one stimulus electrode generates a pre-determined, electrodynamic field that is applied to biological matter; and
sensing a response electrodynamic field by means of at least one sensing electrode and providing a corresponding electric response signal to a sensing circuit,
wherein the electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

The present invention is advantageous because a plurality of different pseudo-random binary sequences providing a pre-determined, electrodynamic field with a plurality of different ranges of frequencies enable a greater breadth of frequencies for a given sequence length and data rate compared to a single pseudo-random binary sequence, which has a breadth of frequencies that is linearly proportional to its length. The present invention therefore enables a greater bandwidth for the biological measurement apparatus relative to using a pseudo-random binary sequence with one range of frequencies.

In particular, in order to achieve a pseudo-random noise signal including frequencies across several orders of magnitude of bandwidth with a single pseudo-random binary sequence, it would require the sequence length to be very long, which would be difficult to send to a host machine and it would be computationally intensive to perform post processing. Furthermore, the greater frequency bandwidth enabled by the present invention also enables a log distributed series of frequency measurements, rather than only linearly distributed measurements, which is advantageous because a linearly distributed series of frequency measurements can cause over representation of higher frequencies and under representation of lower frequencies.

The use of a plurality of different pseudo-random binary sequences may provide advantages in other apparatus, where a broadband system response is required.

Hence, according to a further aspect of the invention, there is provided measurement apparatus comprising:
a stimulation apparatus configured to provide an electric stimulus signal,
a sensing apparatus provide a corresponding electric response signal,
wherein the electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

According to a further aspect of the invention, there is provided a method of measurement, the method comprising:
providing an electric stimulus signal to at least one stimulus electrode, such that the at least one stimulus electrode generates a pre-determined, electrodynamic field; and
sensing a response electrodynamic field by means of at least one sensing electrode and generating a corresponding electric response signal,
wherein the electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

The electric stimulus signal and hence the pre-determined, electrodynamic field may comprise a signal packet including the plurality of different pseudo-random binary sequences. One or more signal packets of the electric stimulus signal and hence the pre-determined, electrodynamic field may be applied to the material to be measured. Where more than one signal packet of the electric stimulus signal and hence the pre-determined, electrodynamic field is applied to the material to be measured, the signal packets may be applied sequentially.

The pre-determined, electrodynamic field may be applied to a material to be measured, which may comprise a fluent material or a particle, eg a biological particle, carried by a fluent material. The measurement apparatus may comprise, or be adapted to be operative with, a flow apparatus that provides for flow of the material to be measured relative to the stimulation apparatus and the sensing apparatus. Where the material to be measured comprises particles, the flow apparatus may be configured to present one particle at a time to be measured. At least one signal packet of the electric stimulus signal and hence the pre-determined, electrodynamic field may be applied to each particle to be measured or each volume of fluent material to be measured.

The present invention is particularly advantageous when used as biological measurement apparatus in Process Analytics Technology (PAT), as it may enable continual monitoring of cell cultures whilst they are within a bioreactor and may therefore enable more reliable and/or more detailed data to be obtained relative to prior art monitoring systems.

The electric stimulus signal may comprise three or more pseudo-random binary sequences, each pseudo-random binary sequence having a length and data rate that provides the pre-determined, electrodynamic field with a range of frequencies that is different to the ranges of frequencies provided by the other pseudo-random binary sequences.

The measurement apparatus may be configured to generate the pseudo-random noise signal. The pseudo-random noise signal may be generated from a pseudo-random binary sequence. The electric stimulus signal provided by the stimulation circuit may comprise a pseudo-random binary signal. The pseudo-random binary sequences are generated with a deterministic algorithm. The pseudo-random binary sequence nevertheless exhibits statistical behaviour similar to a truly random sequence. The pseudo-random binary sequence may be a maximum length sequence, a so-called “m-sequence”, which may be generated by a linear feedback shift register. Where a binary sequence is generated by a linear feedback shift register, the output eventually repeats itself. An m-sequence is the longest possible non-repeating sequence for a given number of shift registers.

The pseudo-random noise signal may therefore comprise an m-sequence. M-sequences exhibit a flat power spectral density across a desired bandwidth of operation. Furthermore, m-sequences may be readily provided for by way of standard digital circuitry and thus may be suited to implementation in an integrated circuit formed by a semiconductor fabrication process, such as CMOS.

The stimulation circuit may be operative to generate a stimulation signal in the form of an m-sequence by way of a linear feedback shift register or otherwise as would be within the ordinary design skills of the person skilled in the art. For example, the stimulation circuit may comprise memory storing the m-sequences. Alternatively, an m-sequence may be provided by an external signal generator.

The plurality of pseudo-random binary sequences may be m-sequences. The length of each m-sequence is 2ⁿ−1, where n is the number of registers in the linear-feedback shift register. The plurality of pseudo-random binary sequences may have the same length or different lengths. The range of frequencies provided by each pseudo-random binary sequence may be determined by the length of each of the pseudo-random binary sequence, determined in numbers of bits, and the bit rate of the electric stimulus signal.

The range of frequencies of each pseudo-random binary sequence is determined by the binary signal achievable with the length of the pseudo-random binary sequence and the bit rate of the electric stimulus signal. The highest frequency of the range of frequencies may be an alternating sequence of bits, immediately adjacent to each other, which will have a frequency of half of the bit rate. The lowest frequency may be a constant value across the entire length of the sequence, which will have a frequency of the bit rate divided by the length of the sequence. The same clock may be used for generation of the plurality of pseudo-random binary sequences. The bit rate of the plurality of pseudo-random binary sequences may be different, however, by the bit period of the lower frequency pseudo-random binary sequence being a whole-number multiple of the bit period of the higher frequency pseudo-random binary sequence.

An n-register m-sequence will have a length of 2ⁿ−1 bits. At a data rate of f, the n-register m-sequence will provide frequencies of f/2 to f/(2ⁿ−1). For example, a 5-register m-sequence will have a length of 31 bits. In an example provided for illustrative purposes, at a data rate of 950 MHz, the 5-register m-sequence will provide frequencies of 475 MHz to 30.65 MHz. In contrast, a 7-register m-sequence will have a length of 127 bits. At a data rate of 50 MHz, the 7-register m-sequence will provide frequencies of 25 MHz to 393 kHz. Hence, using only 158 bits, a frequency range of 475 MHz to 393 kHz is provided. For comparison, an 11-register m-sequence having a length of 2,048 bits would be required, at a data rate of 950 MHz, to provide frequencies of 475 MHz to 464 kHz, extending across the same orders of magnitude as the aforementioned example of the present invention, but requiring a length that is an order of magnitude greater than is required for the present invention.

The plurality of pseudo-random binary sequences may comprise at least a lower frequency pseudo-random binary sequence and a higher frequency pseudo-random binary sequence.

Each pseudo-random binary sequence may provide a sub frame in a frame of the electric stimulus signal. The plurality of pseudo-random binary sequences may be separated from each other in time by a period, which may be at least one m-sequence period.

The measurement apparatus may comprise processing apparatus, which may include the stimulation circuit and/or the sensing circuit. The processing apparatus and/or the sensing circuit may be configured to receive the electric response signal and to convert the electric response signal to digital form. The processing apparatus and/or the sensing circuit may therefore have an analogue input and a digital output, such that the output is a digital response signal.

The processing apparatus and/or the sensing circuit may therefore comprise an analogue-to-digital converter and whatever signal conditioning circuitry may be required, such as an amplifier and an anti-aliasing filter. The processing apparatus may be constituted by any suitable electronic apparatus, such as a separate analogue-to-digital converter circuit, a separate amplifier circuit and a separate filter circuit or a configurable integrated circuit, such as an FPGA, or a dedicated integrated circuit, such as an Application Specific Integrated Circuit (ASIC), comprising such circuits.

The digital response signal may have a frame size, which may have a pre-determined length, determined by the total number of bits in the frame, that accommodates the digital response signal from both the plurality of pseudo-random binary sequences. The digital response signal from the first and second pseudo-random binary sequences may therefore constitute respective sub frames of the digital response signal.

The same clock may be used for the generation of the electric stimulus signal, comprising the plurality of pseudo-random binary sequences, and the sample rate of the processing apparatus. This ensures that the electric stimulus signal does not drift in time relative to the digital response signal. The sample rate of the processing apparatus may, however, be different to the bit rate of the electric stimulus signal, as discussed in more detail below. The sample rate of the processing apparatus and the bit rate of the electric stimulus signal may also be expressed in terms of the sample period of the processing apparatus and the bit period of the electric stimulus signal.

The digital response signal may have a bit rate that is equal to the bit rate of the electric stimulus signal. The period of each bit of the digital response signal from the lower frequency pseudo-random binary sequence may be a whole number multiple of each bit of the digital response signal from the higher frequency pseudo-random binary sequence.

The processing apparatus may have a bandwidth that is sufficient for the range of frequencies being measured. However, the processing apparatus may nevertheless have a sample rate that is lower than the bit rate of the electric response signal, and hence lower than the highest frequency of the higher frequency pseudo-random binary sequence. The processing apparatus may therefore have a sample period that is higher than the bit period of the electric response signal. Although it may be possible to use processing apparatus having a sample rate that is at least equal to the bit rate of the electric response signal, the resultant high data rate may be problematic.

With appropriate selection of the bit period of the electric stimulus signal and the sample period of the processing apparatus, the processing apparatus may be configured to sample the electric response signal at a lower rate than the bit period, but over multiple interleaved passes, which together provide a complete sampling of the electric response signal. This enables processing apparatus with a sample rate that is lower than the bit rate of the electric response signal to provide a digital response signal with a data rate that is greater than the sample rate of the processing apparatus.

In a particular embodiment, the processing apparatus is configured with a sample period that equals a whole number multiple of the bit periods of the electric response signal minus a whole-number fraction of the bit period, and the processing apparatus is configured to sample the electric response signal in a number of interleaved passes.

In respect of the first aspect of the invention, this configuration may be used to sample a higher frequency component of the electric response signal, ie that part that corresponds to a higher frequency pseudo-random binary sequence.

According to a further aspect of the invention, there is provided biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the stimulus electrodes generate a pre-determined, electrodynamic field that is applied to biological matter,
a sensing apparatus including at least one sensing electrode and a sensing circuit, the at least one sensing electrode configured to sense the response electrodynamic field and provide a corresponding electric response signal having a bit period to the sensing circuit, and
a processing apparatus configured to receive the electric response signal and to convert the electric response signal to a digital response signal,
wherein processing apparatus is configured with a sample period that equals a whole number multiple of the bit periods of the electric response signal plus or minus a whole-number fraction of the bit period, and the processing apparatus is configured to sample the electric response signal in a number of interleaved passes.

According to a further aspect of the invention, there is provided a method of biological measurement, the method comprising:
providing an electric stimulus signal to at least one stimulus electrode, such that the at least one stimulus electrode generates a pre-determined, electrodynamic field that is applied to biological matter,
sensing a response electrodynamic field by means of at least one sensing electrode and generating a corresponding electric response signal having a bit period, and
converting the electric response signal to a digital response signal using processing apparatus,
wherein the processing apparatus is configured with a sample period that equals a whole number multiple of the bit periods of the electric response signal plus or minus a whole-number fraction of the bit period, and the electric response signal is sampled in a plurality of interleaved passes.

By “whole-number fraction” is meant a fraction that is equal to the inverse of a whole number.

The biological measurement apparatus according to this aspect of the invention is advantageous because processing apparatus having a sample rate that is lower than the bit rate of the electric response signal may be configured to provide an effective sample rate that is equal to or greater than the bit rate of the electric response signal.

The use of this configuration may provide advantages in other apparatus, for example where a broadband system response is required. Hence, according to a further aspect of the invention, there is provided measurement apparatus comprising:
a stimulation apparatus configured to provide an electric stimulus signal,
a sensing apparatus provide a corresponding electric response signal having a bit period, and
a processing apparatus configured to receive the electric response signal and to convert the electric response signal to digital form,
wherein the processing apparatus is configured with a sample period that equals a whole number multiple of the bit periods of the electric response signal minus a whole-number fraction of the bit period, and the processing apparatus is configured to sample the electric response signal in a plurality of interleaved passes.

According to a further aspect of the invention, there is provided a method of measurement, the method comprising:
providing an electric stimulus signal to at least one stimulus electrode, such that the at least one stimulus electrode generates a pre-determined, electrodynamic field;
sensing a response electrodynamic field by means of at least one sensing electrode and generating a corresponding electric response signal having a bit period, and
converting the electric response signal to a digital response signal using processing apparatus,
wherein the processing apparatus is configured with a sample period that equals a whole number multiple of the bit periods of the electric response signal plus or minus a whole-number fraction of the bit period, and the electric response signal is sampled in a plurality of interleaved passes.

For example, the processing apparatus may be configured with a sample period that equals the bit period of the electric response signal multiplied by n, minus 1/n of the bit period, and the processing apparatus is configured to sample the electric response signal in a plurality of interleaved passes. This configuration provides an oversample ratio of n, increasing the highest frequency achievable by the apparatus by a multiple of n.

Where the electric stimulus signal comprises a first pseudo-random binary sequence having a length and data rate that provide the pre-determined, electrodynamic field with a first range of frequencies, and the electric stimulus signal comprises a second pseudo-random binary sequence having a length and data rate that provide the pre-determined, electrodynamic field with a second, different range of frequencies, this sampling configuration may be used to sample the higher frequency component of the electric response signal, ie that part that corresponds to the higher frequency pseudo-random binary sequence.

When sampling the electric response signal in respect of a lower frequency pseudo-random binary sequence, a lower sample rate may be desired than that used for a higher frequency pseudo-random binary sequence, eg lower by a factor of n. This may be achieved by under sampling, eg by discarding all but 1 in n samples, or all of the samples could be averaged. However, a preferred configuration is to discard only those samples that align with a transition point of the pseudo-random binary sequence, and the remaining n−1 samples being averaged to provide the digital response signal.

According to a further aspect of the invention, there is provided biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the stimulus electrodes generate a pre-determined, electrodynamic field that is applied to biological matter,
a sensing apparatus including at least one sensing electrode and a sensing circuit, the at least one sensing electrode configured to sense the response electrodynamic field and provide a corresponding electric response signal having a bit period to the sensing circuit, and
a processing apparatus configured to receive the electric response signal and to convert the electric response signal to a digital response signal,
wherein the processing apparatus has a sample rate that is a multiple n greater than lower frequencies of the electric response signal, and the processing apparatus is configured to discard samples that align with a transition point of the pseudo-random binary sequence, and to average the remaining n−1 samples to provide the digital response signal.

According to a further aspect of the invention, there is provided a method of biological measurement, the method comprising:
providing an electric stimulus signal to at least one stimulus electrode, such that the at least one stimulus electrode generates a pre-determined, electrodynamic field that is applied to biological matter,
sensing a response electrodynamic field by means of at least one sensing electrode and generating a corresponding electric response signal having a bit period, and
converting the electric response signal to a digital response signal using processing apparatus,
wherein the processing apparatus has a sample rate that is a multiple n greater than lower frequencies of the electric response signal, and the samples that align with a transition point of the pseudo-random binary sequence are discarded, and the remaining n−1 samples are averaged to provide the digital response signal.

The biological measurement apparatus according to this aspect of the invention is advantageous because this configuration increased the signal to noise ratio (SNR) of the digital response signal relative to discarding all but 1 in n samples, whilst also avoiding the skewing of data at high frequencies that can be caused by averaging each n samples without the need for computationally intensive adjustments.

The use of this sampling configuration may provide advantages in other apparatus, where a broadband system response is required. Hence, according to a further aspect of the invention, there is provided measurement apparatus comprising:
a stimulation apparatus configured to provide an electric stimulus signal,
a sensing apparatus provide a corresponding electric response signal, and
a processing apparatus configured to receive the electric response signal and to convert the electric response signal to digital form,
wherein the processing apparatus has a sample rate that is a multiple n greater than the bit period of the lower frequencies of the electric response signal, and the processing apparatus is configured to discard samples that align with a transition point of the pseudo-random binary sequence, and to average the remaining n−1 samples to provide the digital response signal.

Where the electric stimulus signal comprises a first pseudo-random binary sequence having a length and data rate that provide the pre-determined, electrodynamic field with a first range of frequencies, and the electric stimulus signal comprises a second pseudo-random binary sequence having a length and data rate that provide the pre-determined, electrodynamic field with a second, different range of frequencies, this sampling configuration may be used to sample the lower frequency component of the electric response signal, ie that part that corresponds to the lower frequency pseudo-random binary sequence.

In addition, where the processing apparatus has a sample period that equals the bit period of the electric response signal, the processing apparatus may be configured with a sample rate that is a multiple n greater than the bit period of the electric response signal, where n is two or greater.

The digital response signal may be stored in memory, eg a single section of memory. The digital response signal may be output at a data rate that is equal to the bit rate of the higher frequency pseudo-random binary sequence of the electric stimulation signal. The period of each bit of the digital response signal from the lower frequency pseudo-random binary sequence may be a whole number multiple of each bit of the digital response signal from the higher frequency pseudo-random binary sequence. Each bit of the digital response signal for the lower frequency pseudo-random binary sequence may therefore be configured to have a period equal to the whole number multiple of each bit of the digital response signal from the higher frequency pseudo-random binary sequence. The processing apparatus and/or the sensing circuit may comprise a processor and may be operative to decode the received output signal, which may be m-sequence encoded data.

The impulse response may be calculated by application of a Hadamard transform, such as the Fast Hadamard transform. This provides for rapid calculation of the impulse response. The fast Hadamard transform is given by:

$${Ψ}^{′} = \frac{1}{{2}^{m - 1}} {ξ}_{1} H {ξ}_{2} η$$

where Ψ′ is the estimated output spectrum of the system under test, m is the sequence order, H is the Hadamard matrix, q is the measured m-sequence encoded response, and ξ₁ and ξ₂ are the encode and decode matrices for transforming m-sequence data into the correct order for use with the Hadamard matrix. In one form, ξ₁ and ξ₂ are equal to each other.

Alternatively, analysis apparatus may be configured to decode the received output signal, which may be m-sequence encoded data, for example by cross-correlation. The analysis apparatus may be constituted by any suitable electronic apparatus, e.g. a general purpose computer, such as a Personal Computer (PC).

The analysis apparatus may be further operative to perform a Fourier Transform, such as a Fast Fourier Transform (FFT), on the decoded output signal to thereby provide frequency domain data. The frequency domain data may be displayed for user interpretation. The frequency domain data may provide for characterisation of the biological cells in the analyte, such as in respect of their dimensions and composition whereby the nature of a particular cell type can be determined or different cell types can be differentiated.

The measurement apparatus may be operative to measure one or more biological cells held in a sample holder to determine properties of the cell or cells. It is known to constitute the measurement apparatus as an integrated circuit, such as a CMOS integrated circuit. One known form of integrated circuit measurement apparatus comprises an electrode array. In its simplest form the electrode array is one dimensional whereby there is a line of electrodes over which a cell flows or is moved, or cells flow or are moved. In a more complex form, the electrode array is two dimensional to thereby define plural parallel paths whereby more biological cells can be measured simultaneously. Irrespective of the complexity of the electrode array, an electrode in the array defines a complex measurement capacitor (CM) with a common electrode or with another electrode in the array, and with the biological cell and the fluid medium between these two electrodes, acting as the dielectric.

The electrodes may be generally in cuboidal, and the operative surfaces of the electrodes may be square or rectangular. The sensing electrodes and the stimulation electrodes may be arranged with their operative surfaces in a generally planar arrangement, ie in the same plane or parallel planes. The sensing electrodes and the stimulation electrodes may be arranged side-by-side. The operative surfaces of the electrodes may be flat. The sensing electrodes and the stimulation electrodes may be provided on an exposed surface of a semiconductor, and may be formed in an integrated circuit formed by a semiconductor fabrication process, such as CMOS.

The measurement apparatus further comprises an output buffer for each measurement capacitor. The output signal is processed to determine one or more properties of a biological cell sensed by the measurement capacitor.

The measurement apparatus may comprise, or be adapted to be operative with, a flow apparatus that provides for flow of the material under test, eg the fluent material and the biological particles, relative to the stimulation apparatus and the sensing apparatus.

The stimulation apparatus may comprise at least one electrode pair. A pair of electrodes may be disposed relative to each other and relative to fluent material in the flow apparatus so as to apply an electric field to the fluent material. One of the pair of electrodes may be disposed on or towards a side of the fluent material, such as in a direction perpendicular to a direction of flow of the fluent material. The other of the pair of electrodes may be disposed on or towards a side of the fluent material. The pair of electrodes may be disposed on or towards substantially a same side of the fluent material. The pair of electrodes may be disposed side by side. Such a same side disposition may be appropriate where the particle stimulation apparatus is comprised in a planar semiconductor integrated circuit, such as a CMOS integrated circuit.

In use, a sample of fluent material, such as a biological sample, is introduced into a flow apparatus with the flow apparatus being configured to contain and provide for flow of the fluent material. The flow apparatus may be configured to contain the fluent material so as to provide for flow of the fluent material. The flow apparatus may, for example, define an open-ended channel which contains the fluent material and allows for flow such as when flow is created by way of a pump. Alternatively or in addition, the flow apparatus may be configured to actuate flow of itself. More specifically the flow apparatus may be configured to draw the fluent material through the flow apparatus by way of capillary action.

The measurement apparatus may comprise an integrated circuit formed by a semiconductor fabrication process, such as a CMOS fabrication process. The particles may be biological particles, e.g. cells or other structures including viruses, multi-cell organisms, bacteria and spores. Sensing of particles may provide for analysis of the particles involving, for example, differentiation of cell species from each or one another.

The measurement apparatus may be configured to measure a quantity impacted by electrical permittivity of a particle. The measurement apparatus may be configured to measure a quantity impacted by at least one of real and imaginary electrical permittivity of a particle. The quantity being measured may be the change to an applied electrodynamic field, ie a stimulus signal, caused by the presence of a particle.

A particle, such as a cell, may interact with the applied electrodynamic field in a characteristic fashion so as to provide a response that may be measured. The measurement apparatus may therefore be used to measure characteristic of a biological particle in fluent material. Furthermore, different biological particles may cooperate with the applied electrodynamic field in different characteristic fashions so as to provide corresponding different responses. The measurement apparatus may therefore be used to characterise a biological particle. For example, the measurement apparatus may be used to at least one of differentiate one type of cell from another type of cell and determine a characteristic of a cell, such as a dimension of the cell or a composition of a cell.

The at least one stimulation electrode and the at least one sensing electrode may be arranged for impedance or dielectric spectroscopy. At least one stimulation electrode and at least one sensing electrode may be arranged to form a capacitor, with the fluent material and any biological particle therein being located in the electric field between the electrodes. Where the at least one stimulation electrode and the at least one sensing electrode are arranged with their operative surfaces in a generally planar arrangement, side-by-side, the fluent material and any biological particle therein may flow over the electrodes.

A pair of stimulation and sensing electrodes may be disposed relative to each other and relative to fluent material in the flow apparatus so as to sense an electric field, ie the electrical response signal, present in the fluent material. One of the pair of electrodes may be disposed on or towards a side of the fluent material, such as in a direction perpendicular to a direction of flow of the fluent material. The other of the pair of electrodes may be disposed on or towards a side of the fluent material. The pair of electrodes may be disposed on or towards substantially a same side of the fluent material. The pair of electrodes may be disposed side by side. Such a same side disposition may be appropriate where the measurement apparatus is comprised in a planar semiconductor integrated circuit, such as a CMOS integrated circuit.

An electrode may have a dimension, such as at least one of width and height, of less than substantially 100 microns, 50 microns, 30 microns, 20 microns, 15 microns, 10 microns, 5 microns, 3 microns or 1 micron. Alternatively or in addition, an electrode may have a dimension of more than substantially 0.5 microns, 1 micron, 5 microns, 10 microns, 15 microns, 20 microns, 30 microns or 50 microns.

The measurement apparatus may comprise plural spaced apart sensing electrodes, e.g. plural sensing electrode pairs, with each sensing electrode being operative to sense particles. Furthermore, the flow apparatus may define a flow path along which a particle travels and the measurement apparatus may be configured such that as a particle travels along the flow path the electric response field of the particle is sensed by successive sensing electrodes, eg two sensing electrodes for a differential measurement.

The sensing circuit may be configured to sense charge, such as may be present on a sensing electrode. More specifically, the sensing circuit may comprise a capacitor which is operative to sense charge and to convert the sensed charge to a voltage signal. The sensing circuit may comprise a high impedance input to thereby provide for a significant sensed signal. More specifically the sensing circuit may comprise an impedance buffer, such as a field-effect transistor (FET). A FET may provide a capacitive load, e.g. for a sensing electrode of the sensing apparatus. Alternatively or in addition, the sensing circuit may provide one of a voltage signal and a current signal as the output signal.

Alternatively or in addition, the sensing circuit may be configured to amplify the input signal. For example, the sensing circuit may be configured to convert electrical charge present on a sensing electrode to a corresponding voltage and to amplify the converted voltage; or to a corresponding current and to amplify the converted current.

The measurement apparatus may be configured to compare the electric stimulus signal with the electric response signal, e.g. as sensed by the sensing apparatus. Comparison of the electric stimulus signal with the electric response signal may comprise cross-correlation of the electric stimulus signal with the electric response signal. The measurement apparatus may be operative to determine a time delay between application of the electric stimulus signal and the response as provided by at least one particle in the fluent material. The measurement apparatus may be further operative to determine a transfer function or at least to approximate a transfer function for the at least one particle in dependence on the time delay. The determined or approximated transfer function may then provide for characterisation of the at least one particle.

The pre-determined, electrodynamic field applied to the biological particle may comprise at least one corresponding frequency component. The sensing apparatus may be configured to sense a frequency component comprised in the response electrodynamic field, sensed by the particle sensing apparatus, the frequency component being at least 1 kHz, 10 kHz, 50 kHz, 100 kHz, 250 kHz, 500 kHz, 1 MHz, 5 MHz, 10 MHz, 25 MHz, 50 MHz, 75 MHz, 100 MHz, 250 MHz, 500 MHz, 750 MHz, 1 GHz, 1.25 GHz, 1.5 GHz, 1.75 GHz, 2 GHz. 2.5 GHz, 2.75 GHz or 5 GHz. More specifically the frequency component may be between 10 kHz and 5 GHz. Under certain circumstances, at a frequency above 1 MHz the measurement apparatus may be operative to characterise an inside of the at least one particle, such as where the at least one particle is a biological cell. Under certain circumstances, a frequency above 1 kHz may be required to characterise a particle in respect of its external characteristics, such as a size of a biological cell.

The sensing apparatus of the measurement apparatus may comprise an array of sensing electrodes, such as an array of sensing electrodes as described elsewhere herein. The array may extend in a direction of flow of fluent material as provided by the flow apparatus. A particle may therefore be sensed by a succession of sensing electrodes as the particle progresses through the flow apparatus.

Alternatively or in addition, the array may extend in a direction perpendicular to a direction of flow of fluent material through flow apparatus. More specifically, the array may extend in the direction of flow and perpendicular to the direction of flow whereby the array is a two dimensional array. An array of sensing electrodes may provide for simultaneous sensing operations.

The measurement apparatus may be operative and perhaps also configured to be label free. The biological sensing apparatus may therefore operate on fluent material lacking any label, such as a fluorochrome or microbeads.

The measurement apparatus may be operative and perhaps also configured for sensing of microbiological samples. The measurement apparatus may therefore be configured to measure particles, such as biological elements, which have a dimension of less than substantially 500 microns, 250 microns, 200 microns, 150 microns, 100 microns, 50 microns, 25 microns, 10 microns, 5 microns, 2 microns, 1 micron, 500 nm, 250 nm, 100 nm, 50 nm, 25 nm, 10 nm or 5 nm. Alternatively or in addition, the measurement apparatus may be configured to measure particles which have a dimension greater than substantially 2 nm, 5 nm, 10 nm, 25 nm, 50 nm, 100 nm, 250 nm, 0.5 microns, 1 micron, 2 microns, 5 microns, 10 microns, 25 microns, 50 microns, 100 microns, 150 microns, 200 microns or 250 microns.

The measurement apparatus may be configured for a particular size of particle or a range of sizes of particles in respect of a dimension of at least one of the stimulation apparatus and the sensing apparatus, such as a size of at least one electrode. More specifically, a dimension of at least one of the stimulation apparatus and the sensing apparatus may correspond to a size of particle or a range of sizes of particles.

The semiconductor fabrication process may be a planar semiconductor fabrication process. Alternatively or in addition, the semiconductor fabrication process may be a metal-oxide semiconductor process, such as a CMOS process. Alternatively or in addition, the semiconductor fabrication process may be a submicron semiconductor fabrication process, such as a 0.18 micron CMOS process and perhaps a high voltage 0.18 micron CMOS process.

The fluent material may be substantially liquid, e.g. at room temperature. The fluent material may therefore comprise a liquid which carries the particles. More specifically, the fluent material may comprise charge carriers, such as salt molecules. For example the fluent material may comprise phosphate buffered saline (PBS).

The flow apparatus may define a main channel through which the fluent material flows when in use. The sensing apparatus may be disposed relative to the main channel so as to provide for sensing of particles present in the main channel. The sensing apparatus may be disposed on at least one of first and second opposite sides of a flow of fluent material. Thus, for example, components such as sensing electrodes of the sensing apparatus may be disposed on one side of the flow of fluent material. According to another example, components of the particle sensing apparatus may be disposed on both sides of the flow of fluent material.

The flow apparatus may comprise a sample inlet which is configured to receive a sample of fluent material, e.g. by way of injection, which is to be subject to measurement, the sample inlet being in fluid communication with the main channel. The flow apparatus may comprise a sample outlet at an opposite end of the flow apparatus from the sample inlet, the sample outlet being in fluid communication with the main channel. The sample outlet may provide for flow of fluent material from the main channel.

The flow apparatus may comprise at least one further inlet disposed laterally of the sample inlet. More specifically, the flow apparatus may comprise first and second further inlets with the first inlet disposed laterally on one side of the sample inlet and the second inlet disposed laterally on another opposite side of the sample inlet. The at least one further inlet may be in fluid communication with the main channel. In use, a sheath fluid, such as phosphate buffered saline (PBS), may be received by the at least one further inlet to thereby provide for a flow of sheath fluid in the main channel, the flow of sheath fluid being lateral of a flow of fluent material. The flow of sheath fluid may provide for registration of the particle comprising fluent material with the particle sensing apparatus and may also help preserve the integrity of the flow of fluent material as it progresses though the flow apparatus.

The flow apparatus may be formed from glass and/or at least in part from a polymer, such as poly(methyl methacrylate) (PMMA). The flow apparatus may be of a length of substantially 25 mm and of a width of substantially 10 mm. The stimulation and sensing apparatus may be formed separately from the flow apparatus. The stimulation and sensing apparatus may be disposed relative to the flow apparatus by attaching the stimulation and sensing apparatus and the flow apparatus to each other. The stimulation and sensing apparatus and the flow apparatus may be adhered together, for example by appropriate chemical or physical bonding, or may be mechanically attached to each other, e.g. by way of a fastener apparatus comprising a silicone gasket layer, which may be releasable. Appropriate chemical or physical bonding may include plasma bonding.

The biological measurement apparatus may be configured to be operable as a flow cytometer. The measurement apparatus may further comprise control apparatus. The control apparatus may be constituted by any suitable electronic apparatus, such as a microprocessor or a configurable electronic circuit, such as a Field Programmable Gate Array (FPGA).

The measurement apparatus may comprise flow inducing apparatus, i.e. a pump, which is operative to induce a flow of fluent material through the flow apparatus. The flow inducing apparatus may be controlled, for example in respect of a rate of flow of fluent material through the flow apparatus, in dependence on an output from the sensing apparatus. The control apparatus may be operative to receive an output from the sensing apparatus and to provide an output to the flow inducing apparatus in dependence thereon.

The sensing apparatus as described elsewhere herein may be operative to provide for determination of a rate of flow of fluent material through the flow apparatus, the rate of flow being received by the control apparatus. Where the sensing apparatus comprises plural spaced apart sensing electrodes with each sensing electrode being operative to sense particles, the rate of flow may be determined in dependence on the separation between the sensing electrodes being known and a time between sensing of a particle by different sensing electrodes.

Alternatively or in addition, characterisation of at least one particle as described elsewhere herein may be compared with a predetermined criterion and the flow inducing apparatus may be controlled in dependence on the comparison. For example, characterisation of the at least one particle may comprise a level of confidence value which is compared with a predefined value. More specifically, if the level of confidence value is below the predefined value the flow inducing apparatus may be operative to reduce a rate of flow of the fluent material to thereby provide for improved characterisation.

The processing apparatus may provide an output signal, eg a digital output signal. The output signal may be stored in memory of the measurement apparatus. The measurement apparatus may comprise an output for sending the output signal to analysis apparatus, which may or may not be integrated with the measurement apparatus.

The analysis apparatus may be configured to make determinations with regards to particles comprised in the fluent material in dependence on at least one output from the particle sensing apparatus. For example, the analysis apparatus may be operative to make determinations in dependence on electric field measurements made by the particle sensing apparatus after analogue to digital conversion. Determinations may be made in respect of the like of the density of particles comprised in the fluent material, differentiation of one form of particle from another and characteristics of particles, such as in respect of dimensions or composition. The analysis apparatus may be constituted by any suitable electronic apparatus, e.g. a general purpose computer, such as a Personal Computer (PC), an embedded microprocessor, a configurable electronic circuit, such as a FPGA or the like.

The sensor chip may have orthogonal dimensions that are each less than 50 mm, less than 20 mm or less than 10 mm, eg 5 mm×5 mm. The sensor chip may be configured to be mounted within a bioreactor, eg from microbioreactors to large bioreactors. Microbioreactors are particularly suitable for use in personalised medicine, eg small batches may be required on a per patient basis.

Further features and advantages of the present invention will become apparent from the following specific description, which is given by way of example only and with reference to the accompanying drawings, in which:

FIG. 1 is a block diagram representation of biological measurement apparatus according to an embodiment of the present invention;

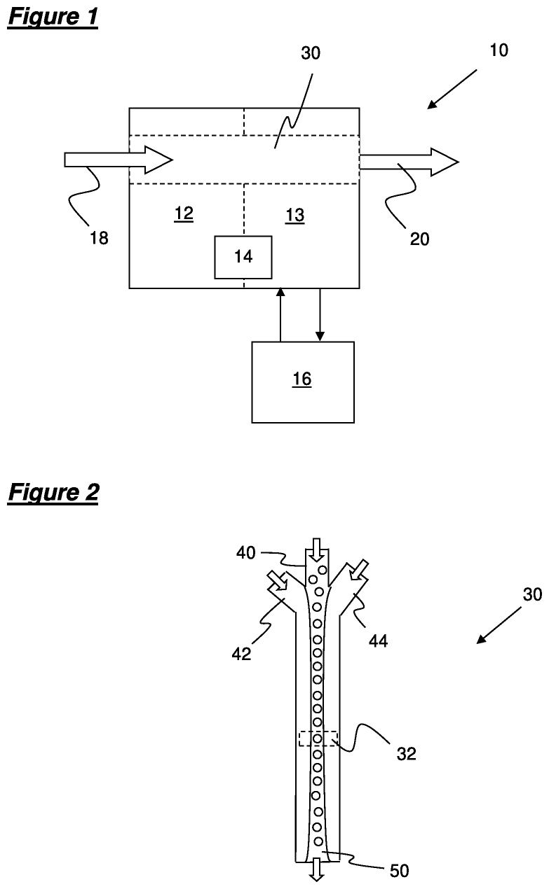

FIG. 2 is a representation of a flow apparatus that is part of the biological measurement apparatus embodiment;

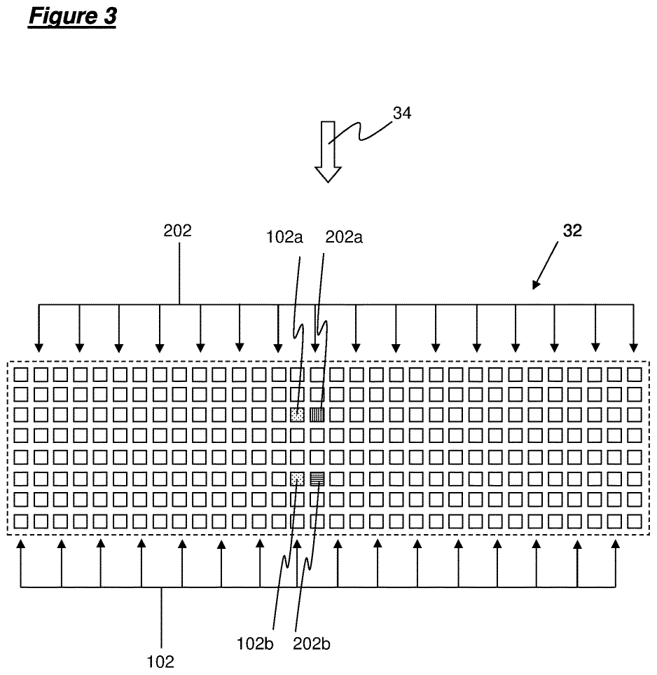

FIG. 3 is a representation of an electrode array that is part of the biological measurement apparatus embodiment;

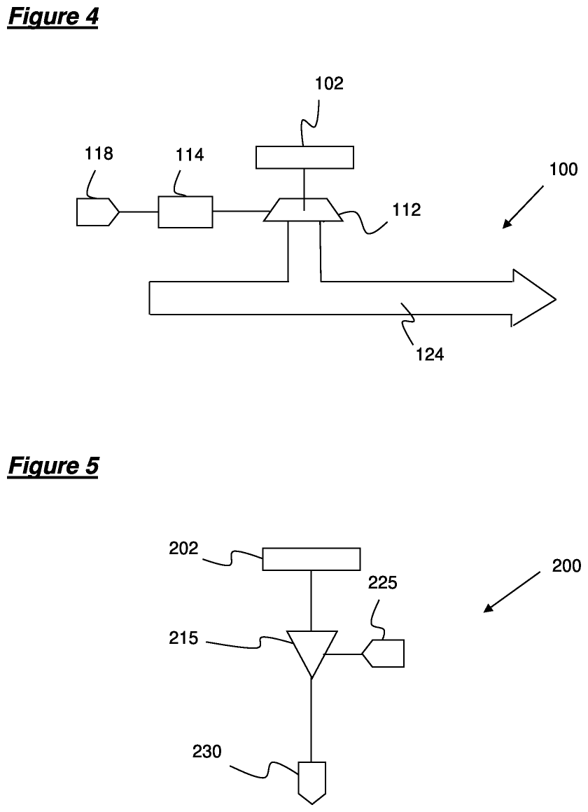

FIG. 4 is a circuit representation of stimulation apparatus that is part of the biological measurement apparatus embodiment; and

FIG. 5 is a circuit representation of sensing apparatus that is part of the biological measurement apparatus embodiment.

A block diagram representation of biological measurement apparatus 10 according to the present invention is shown in FIG. 1. The biological analysis apparatus 10 comprises flow apparatus 30, stimulation apparatus 12, sensing apparatus 13, control and processing apparatus 14, and analysis apparatus 16. The stimulation apparatus 12 and sensing apparatus 13 receive, via the flow apparatus 30, a flow of analyte in the form of phosphate buffered saline (PBS) 18 (which constitutes fluent material) in which biological cells (which constitute particles) are suspended.

An alternative to PBS may be used on account of the present approach providing for sensing which is independent of the suspension material.

The flow of analyte is directed by the flow apparatus 30 through the stimulation apparatus 12 and sensing apparatus 13 where it is subject to stimulation and sensing, as described in detail below, before exiting 20 from the measurement apparatus 10.

The control and processing apparatus 14 controls the application of stimulation signals to the analyte by the stimulation apparatus 12, and processes signals sensed by the sensing apparatus 13. Processing comprises amplification of sensed signals, analogue to digital conversion of sensed signals and storage of converted sensed signals. Although not shown in FIG. 1, the measurement apparatus 10 further comprises a pump which is operative to push or draw analyte through the stimulation apparatus 12 and sensing apparatus 13, via the flow apparatus 30.

The analysis apparatus 16 is operative to make at least one analytical determination in dependence on the stored converted sensed signals. Analytical determinations comprise: detecting the presence of biological cells in the analyte; counting biological cells in the analyte; differentiating one form of biological cell from another; and determining a characteristic of biological cells in the analyte, such as a cell dimension or a cell composition. The analysis apparatus 16 is also operative to provide for supervisory control of the control and processing apparatus 14, e.g. in respect of a change in the form of control of the biological sensing apparatus 12 exercised by the control and processing apparatus 14.

The control and processing apparatus 14 is constituted by any suitable electronic apparatus, such as a separate analogue-to-digital converter circuit, a separate amplifier circuit and a separate electronic memory circuit, or a configurable integrated circuit, such as a System-on-a-Chip (SOC), comprising the digital circuits and an ASIC comprising the analogue circuits. The analysis apparatus 16 is constituted by any suitable electronic apparatus, e.g. a general purpose computer, such as a PC, an embedded microprocessor, a configurable electronic circuit, such as an FPGA, or the like. The control and processing apparatus 14 and analysis apparatus 16 are constituted apart from each other, e.g. as separate modules, or constituted together, e.g. in a same integrated circuit or same general purpose computer.

The flow apparatus 30 receives the analyte 18 and provides for flow of the analyte before the analyte exits 20 from the flow apparatus. With reference to FIG. 2, the measurement apparatus also comprises a two-dimensional array of electrodes 32, which comprise stimulation electrodes of the stimulation apparatus 12 and sensing electrodes of the sensing apparatus 13. The flow apparatus 30 and the array of electrodes 32 are disposed in relation to each other such that the array of electrodes 32 is above a main channel of the flow apparatus 30.

The control and processing apparatus 14 is electrically coupled to the array of electrodes 32. The control and processing apparatus 14 is operative to provide for biological cell stimulation, sensing and actuation by way of the array of electrodes 32.

The arrangement of the array of electrodes is shown in more detail in FIG. 3.

The array of electrodes 32 and the control and processing apparatus 14 are constituted by a CMOS process such as a 0.35 micron CMOS process. The array of electrodes 32 and the control and processing apparatus 14 are both comprised in a CMOS ASIC. Each electrode in the array 32 is 18 microns by 18 microns with a 2 micron gap between electrodes whereby the array pitch is 20 microns. The electrodes are surrounded by a busbar that prevents capacitive coupling to the silicon, and which is grounded.

The thickness and permittivity of the standard polyimide top layer of the ASIC provides insufficient capacitance for proper engagement of the electrodes 32 with the analyte, and hence the fabrication process lacks a polyimide layer deposition step so no polyimide top layer is present. The hydrophilic nature of the silicon nitride layer provides for maximum exposure. However, the silicon nitride layer may be removed over the electrodes, such that the electrodes conduct into the analyte. The ASIC is disposed, as is mentioned above, relative to the flow apparatus 30 such that the array of electrodes 32 engages with the analyte flowing through the flow apparatus 30.

The control and processing apparatus 14 of the ASIC comprises binary to decimal decoders and memory for row and column addressing of the array of electrodes 32, global configuration logic and bias circuitry for the sensor output signal paths. The global configuration logic is operative to provide for the like of memory resetting and the gating of control signals with respect to a global reset signal to ensure all control lines power up in a known state.

The measurement apparatus 10 further comprises a Printed Circuit Board (PCB) which supports and provides for electrical connectivity for electrical circuits which support the ASIC. The electrical circuits comprised in the PCB includes an SOC which is configured to provide various digital functions including the generation of stimulus signals, addressing of individual electrodes in the array of electrodes 32 and communication with a Universal Serial Bus (USB) module.

The SOC 44 is operative to generate a stimulation signal in the form of an m-sequence that is stored in memory and output one bit at a time. In particular, in this embodiment, the stimulation signal consists of a higher frequency m-sequence, which is generated from a linear feedback shift register having 5 registers, and a lower frequency m-sequence, which is generated from a linear feedback shift register having 7 registers. The higher frequency m-sequence therefore has a length of 31 bits and the lower frequency m-sequence has a length of 127 bits. The bit rate of the higher frequency m-sequence of the stimulation signal is 950 Mbps, and the bit rate of the lower frequency m-sequence of the stimulation signal is 50 Mbps.

The PCB includes input signal conditioning circuitry which is configured to receive stimulus signals from the SOC or from the external (un-illustrated) signal generator, and provide for programmable gain amplification of the voltage swing of the stimulus signals.

In addition, the PCB includes output signal conditioning circuitry which performs a variety of functions including fixed gain, low distortion amplification of sensed single ended signals followed by programmable gain amplification or attenuation of such initially amplified signals under the control of a PC.

The output signal conditioning circuitry also includes an analogue-to-digital converter.

The sample rate of the analogue-to-digital converter is 200 MHz. The analogue-to-digital converter is configured to sample the higher frequency m-sequence of the stimulation signal in four interleaved passes, providing a sample length of 124 bits. The theoretical bandwidth of this m-Sequence measurement is therefore 1.9 GHz-30.65 MHz. Due to the ×4 oversample ratio, the flat region of this bandwidth only extends to 475 MHz, ¼ of the theoretical bandwidth, but that through post processing, the higher frequency regions above this point are also accessible.

The analogue-to-digital converter is configured to sample the lower frequency m-sequence of the stimulation signal at 200 MHz, which is four times greater than the bit rate of the lower frequency m-sequence of the stimulation signal. However, the analogue-to-digital converter is configured to discard samples that align with a transition point of the m-sequence, and to average the remaining 3 samples to provide the digital output.

Because the stimulus for both high and low frequency sections of the stimulus signal are from the same source, the low frequency stimulus signal must align to the sampling method. In this case, each low frequency sample period (20 ns) is made up of 4 analogue-to-digital converter samples (5 ns each), as well as 19 high frequency stimulus samples (~1.052 ns each); 19*1.052 ns=20 ns.

To stop significant contamination from one stimulus pattern to the next, a gap of at least one m-sequence period is maintained between the two sections of the capture.

In this specific implementation, the entire stimulus package, which includes both the high and low frequency signals, are stored in a single section of memory. This is then streamed out, bit by bit, at the high frequency data rate (950 MHz in this example). The low frequency signal is created by padding out the (in this case 7-bit) M-Sequence signal by a factor of 19, so each bit lasts for 19 high frequency periods.

As described above, the PCB comprises a USB module. The USB module provides for communication with a PC running software operative to perform the functions of the analysis apparatus 16 of FIG. 1. More specifically, the PC is operative to configure the ASIC 42 and the circuits comprised in the PCB. In addition, the PC receives real-time sensed data or blocks of data which have been acquired and stored locally from the SOC.

The PC is operative to decode the received m-sequence encoded data by cross-correlation to provide the impulse response. The PC is further operative to perform a Fast Fourier Transform (FFT) on the decoded data to thereby provide frequency domain data. The frequency domain data is then displayed for user interpretation. The frequency domain data provides for characterisation of the biological cells in the analyte, such as in respect of their dimensions and composition whereby the nature of a particular cell type can be determined or different cell types can be differentiated.

The PC is also operative to count biological cells present in the analyte and to determine a density of cells present in the analyte in dependence on the flow rate and volume of the flow apparatus with the count and density information being displayed to the user.

A representation of a flow apparatus 30 comprised in the biological sensing apparatus of FIG. 2 is shown in detail in FIG. 2. The flow apparatus 30 of FIG. 2 is formed from glass and has a length of about 25 mm and a width of about 10 mm. The flow apparatus 30 comprises a main channel 34 through which the analyte flows. The array of electrodes 32 is disposed above the main channel 34 so that the electrodes 32 engage with the analyte as the analyte flows through the main channel.

As is described above, the array of electrodes 32 is comprised in a CMOS ASIC. The CMOS ASIC and the flow apparatus 30 are releasably attached to each other by way of a fastener apparatus comprising a silicone gasket layer, such that a proper relative disposition of electrodes and main channel is achieved.

The flow apparatus 30 also comprises a sample inlet 40 which receives the analyte, e.g. by way of injection, and a sample outlet 50 at an opposite end of the flow apparatus from the sample inlet 40. The sample inlet 40 and the sample outlet 50 are each in fluid communication with the main channel 34. In addition, the flow apparatus comprises first and second further inlets 42, 44. The first further inlet 42 is disposed laterally on one side of the sample inlet 40 and the second further inlet 44 is disposed laterally on the other opposite side of the sample inlet. Each of the first and second further inlets 42, 44 are in fluid communication with the main channel 34.

In use, a sheath fluid, such as phosphate buffered saline (PBS), is received by each of the first and second further inlets 42, 44 to thereby provide for a flow of sheath fluid in the main channel, the flow of sheath fluid being lateral of a flow of analyte received by the sample inlet 40. The flow of sheath fluid provides for registration of the biological cell comprising analyte with the array of electrodes 32 and also helps preserve the integrity of the flow of analyte as it progresses though the main channel.

Stimulation and sensing comprises electric field stimulation and electric field sensing. A biological cell cooperates with the applied electric field whereby the electric field is disturbed. Different sizes of a particular type of biological cell will disturb an applied electric field in a different manner. In addition, different types of biological cell will disturb an applied electric field in a different manner. Sensing of the electric field in dependence on the disturbance can therefore provide for detection of the presence of biological cells, determination of relative sizes of cells and differentiation of different types of cell from each or one another.

Electric field stimulation and electric field sensing can both be accomplished within a CMOS ASIC of the form described above. More specifically the array of electrodes 32 is used for both electric field stimulation and electric field sensing, with different sets of electrodes being used for stimulation and sensing.

A stimulation cell 100 configured for single ended operation is shown in FIG. 3. The stimulation cell 100 of FIG. 3 comprises a single electrode 102, which is comprised in the array of electrodes 32. A stimulation signal is applied to the stimulation electrode 102.

The stimulation cell 100 further comprises a multiplexer 112 which provides for one of two states selected in accordance with a state selection bit 118. The stimulation cell 100 also comprises a memory bit 114 that stores the state of the first state selection bit 118. The memory bits 114 are constituted in Static Random Access Memory (SRAM).

The multiplexer of FIG. 3 provides for one of two states. To provide for one of the two states, the electrode is addressed with the address-sensitive first state selection bit 118 and then the first state selection bit 118 is stored as the memory bit 114.

In a first state when the state selection bit 118 is at zero, the electrode 102 is connected by way of a switch to common ground potential. In a second state when the first state selection bit 118 is at one, the electrode 102 is configured for stimulation whereby the electrode receives a stimulation input from a signal bus 124. The signal bus 124 is electrically connected to the part of the control and processing apparatus 14 which is operative to generate stimulation signals, as described above.

Each stimulation electrode 102 in the array of electrodes 32 comprises the multiplexer and memory circuitry shown in FIG. 4.

A sensing cell 200 configured for single ended operation is shown in FIG. 5. The sensing cell 200 of FIG. 4 comprises a single electrode 202, which is comprised in the array of electrodes 32. The sensing cell 200 also comprises an output buffer 215 and an output pin 230.

The electrode 202 is configured for sensing the electrodynamic response field, whereby the electrode 202 is connected to a sensor output pin 230 via the output buffer 215, which is addressed by second state selection bit 225. The sensor output pin 230 is electrically connected to the part of the control and the processing circuitry 34 that is operative to process sensed signals, as described above.

The stimulus electrodes 102 and the sensing electrodes 202 are arranged in a rectangular array of electrodes 32, which is shown schematically in FIG. 3.

The array of electrodes 32 comprises two columns aligned with the flow of analyte, with a first column consisting of stimulus electrodes 102 and a second column consisting of sensing electrodes 202. The rectangular array of electrodes 32 may comprise a transverse arrangement of multiple pairs of the two columns of electrodes, for example an array of 32 columns and 8 rows, with alternating rows of stimulus electrodes 102 and sensing electrodes 202, as shown in FIG. 3.

In the embodiment of FIG. 3, the main channel of the flow apparatus 30 has a width that is sufficient for five columns of electrodes to be disposed within the main channel, with the remainder of the electrodes of the array 32 being outside the main channel of the flow apparatus. The electrodes that are active, in use, are selected to be in the columns, eg two columns, that are generally central in the main channel of the flow apparatus, where there is laminar flow.

The electrodes that are configured for use in FIG. 3 are the stimulation electrodes 102a and 102b, and the sensing electrodes 202a and 202b. In particular, a stimulation signal is provided to stimulation electrode 102a, and the same stimulation signal or a complementary signal of oppositive polarity is provided to stimulation electrode 102b. The stimulation electrode 102a forms a capacitor with the adjacent sensing electrode 202a, with the fluent material and any biological particle therein being located within the electrical field between these electrodes 102a,202a. Similarly, the stimulation electrode 102b forms a capacitor with the adjacent sensing electrode 202b, with the fluent material and any biological particle therein being located within the electrical field between these electrodes 102b,202b.

The output signals from the sensing circuit associated with the sensing electrodes 202a,202b are compared, eg by subtraction, such that a differential measurement is made. In particular, the concentration of biological particles and the flow rate is controlled such that each biological particle in the flow passes over the first pair of electrodes 102a,202a and then the second pair of electrodes 102b,202b. The differential measurement will be zero until a biological particle passes over the first pair of electrodes 102a,202a or the second pair of electrodes 102b,202b, when a differential signal will be output.

The distance between the first pair of electrodes 102a,202a or the second pair of electrodes 102b,202b is selected to be sufficient that the respective electrical fields are separated, such that a biological particle isn't detected by both pairs of electrodes at the same time.

The pump of the measurement apparatus 10 of FIG. 1 is controlled in dependence on at least one of: a rate of flow of analyte through the measurement apparatus 10; and a level of confidence of characterisation of the analyte flowing through measurement apparatus 10. Considering rate of flow of analyte further, the separation between pairs of electrodes in the array is known and the time of travel of biological cells between pairs of electrodes is determined by the control and processing apparatus 14.

The control and processing apparatus 14 is then operative to determine the speed of movement of biological cells through the measurement apparatus 10. The control and processing apparatus 14 is then operative to control the pump in dependence on the determined speed. For example, if the determined speed is below a predetermined value the control and processing apparatus 14 is operative to increase the flow rate by controlling the pump. Considering level of confidence of characterisation of the analyte further, the control and processing apparatus 14 is operative to characterise biological cells and to determine a level of confidence of the characterisation. The control and processing apparatus 14 is further operative to compare the determined level of confidence with a predetermined level and then to control the pump in dependence thereon. If the determined level of confidence is below the predetermined level the control and processing apparatus 14 is operative to reduce the rate of flow by controlling the pump to thereby provide for improved characterisation of the biological cells.

1. A biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the at least one stimulus electrode generates a pre-determined, electrodynamic field that is applied to biological matter,
a sensing apparatus including at least one sensing electrode and a sensing circuit, the at least one sensing electrode configured to sense a response to the applied electrodynamic field and provide a corresponding electric response signal to the sensing circuit,
wherein the electric stimulus signal comprises a plurality of different pseudo-random binary sequences, each pseudo-random binary sequence having a length and data rate that provide the pre-determined, electrodynamic field with a range of frequencies that is different to the range(s) of frequencies provided by the other pseudo-random binary sequence(s).

2. A biological measurement apparatus as claimed in claim 1, wherein the electric stimulus signal comprises three or more pseudo-random binary sequences, each pseudo-random binary sequence having a length and data rate that provides the pre-determined, electrodynamic field with a range of frequencies that is different to the ranges of frequencies provided by the other pseudo-random binary sequences.

3. A biological measurement apparatus as claimed in claim 1 or claim 2, wherein the electric stimulus signal comprises a log distributed series of pseudo-random binary sequences.

4. A biological measurement apparatus as claimed in any preceding claim, wherein the plurality of pseudo-random binary sequences are maximum length sequences (m-sequences).

5. A biological measurement apparatus as claimed in any preceding claim, wherein the electric stimulus signal comprises a signal packet including the plurality of different pseudo-random binary sequences.

6. A biological measurement apparatus as claimed in any preceding claim, wherein the plurality of pseudo-random binary sequences have different sample rates.

7. A biological measurement apparatus as claimed in any preceding claim, wherein a clock is used for the plurality of pseudo-random binary sequences, the clock being the same for each of the pseudo-random binary sequences.

8. A biological measurement apparatus as claimed in claim 7, wherein the plurality of pseudo-random binary sequences comprise at least a lower frequency pseudo-random binary sequence and a higher frequency pseudo-random binary sequence, and the period of each bit of the lower frequency pseudo-random binary sequence is a whole number multiple of each bit of the higher frequency pseudo-random binary sequence.

9. A biological measurement apparatus as claimed in claim 8, wherein the ranges of frequencies provided by the pseudo-random binary sequences are distributed over a global range of frequencies, and the period of each bit of a lower frequency pseudo-random binary sequence is a whole number multiple of each bit of a higher frequency pseudo-random binary sequence.

10. A biological measurement apparatus as claimed in any preceding claim, wherein the plurality of pseudo-random binary sequences provide a plurality of sub frames in a frame of the electric stimulus signal.

11. A biological measurement apparatus as claimed in claim 10, wherein the respective sub frames of the plurality of pseudo-random binary sequences are separated in time by a sub-frame separation period.

12. A biological measurement apparatus as claimed in claim 11, wherein the sub-frame separation period is at least one m-sequence period.

13. A biological measurement apparatus as claimed in any preceding claim, wherein the measurement apparatus comprises processing apparatus, the processing apparatus being configured to receive the electric response signal in analogue form and to convert the electric response signal to a digital response signal.

14. A biological measurement apparatus as claimed in claim 13, wherein the digital response signal has a bit rate that is equal to the bit rate of the electric stimulus signal, and the period of each bit of the digital response signal from a lower frequency pseudo-random binary sequence is a whole number multiple of each bit of the digital response signal from a higher frequency pseudo-random binary sequence.

15. A biological measurement apparatus as claimed in claim 13 or claim 14, wherein a higher frequency component of the electric response signal has a higher frequency bit period, and the processing apparatus is configured with a sample period that equals a whole number multiple of the higher frequency bit periods minus a whole-number fraction of the higher frequency bit period, and the processing apparatus is configured to sample the electric response signal in a number of interleaved passes.

16. A biological measurement apparatus as claimed in claim 15, wherein the processing apparatus is configured with a sample period that equals the higher frequency bit period multiplied by n, minus 1/n of the higher frequency bit period.

17. A biological measurement apparatus as claimed in any one of claims 13 to 16, wherein the lower frequency component of the electric response signal has a lower frequency bit period, and the processing apparatus has a sample rate that is a multiple n greater than the lower frequency bit period, and the processing apparatus is configured to discard samples that align with a transition point of the pseudo-random binary sequence, and to average the remaining n−1 samples to provide the digital response signal.

18. A biological measurement apparatus as claimed in any one of claims 13 to 17, wherein the digital response signal is stored in a single section of memory.

19. A biological measurement apparatus as claimed in any one of claims 13 to 18, wherein the digital response signal is output at a data rate that is equal to the bit rate of a higher frequency pseudo-random binary sequence of the electric stimulation signal.

20. A biological measurement apparatus as claimed in any one of claims 13 to 19, wherein the period of each bit of the digital response signal from a lower frequency pseudo-random binary sequence is a whole number multiple of each bit of the digital response signal from a higher frequency pseudo-random binary sequence.

21. A biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the stimulus electrodes generate a pre-determined, electrodynamic field that is applied to biological matter,
a sensing apparatus including at least one sensing electrode and a sensing circuit, the at least one sensing electrode configured to sense the response electrodynamic field and provide a corresponding electric response signal having a bit period to the sensing circuit, and
a processing apparatus configured to receive the electric response signal and to convert the electric response signal to a digital response signal, wherein processing apparatus is configured with a sample period that equals a whole number multiple of the bit periods of the electric response signal plus or minus a whole-number fraction of the bit period, and the processing apparatus is configured to sample the electric response signal in a number of interleaved passes.

22. A biological measurement apparatus, the biological measurement apparatus comprising:
a stimulation apparatus including at least one stimulation electrode and a stimulation circuit, the stimulation circuit configured to provide an electric stimulus signal to the at least one stimulus electrode, such that the stimulus electrodes generate a pre-determined, electrodynamic field that is applied to biological matter,
a sensing apparatus including at least one sensing electrode and a sensing circuit, the at least one sensing electrode configured to sense the response electrodynamic field and provide a corresponding electric response signal having a bit period to the sensing circuit, and
a processing apparatus configured to receive the electric response signal and to convert the electric response signal to a digital response signal, wherein the processing apparatus has a sample rate that is a multiple n greater than lower frequencies of the electric response signal, and the processing apparatus is configured to discard samples that align with a transition point of the pseudo-random binary sequence, and to average the remaining n−1 samples to provide the digital response signal.
